(19) United States
(12) Patent Application Publication
(10) Pub. No.: US 2017/0085762 A1
(43) Pub. Date: Mar. 23, 2017

(54) ENDOSCOPE SYSTEM

(71) Applicant: OLYMPUS CORPORATION, Tokyo (JP)

(72) Inventors: Tatsuya OBARA, Tokyo (JP); Kazuki HONDA, Tokyo (JP); Mikio INOMATA, Tokyo (JP)

(73) Assignee: OLYMPUS CORPORATION, Tokyo (JP)

(21) Appl. No.: 15/367,656

(22) Filed: Dec. 2, 2016

Related U.S. Application Data
(63) Continuation of application No. PCT/JP2015/079174, filed on Oct. 15, 2015.

(30) Foreign Application Priority Data
Nov. 6, 2014 (JP) 2014-226208

(51) Int. Cl. H04N 5/225; H04N 5/262; A61B 1/06; H04N 5/232; A61B 1/04; A61B 1/00

(52) U.S. Cl. CPC H04N 5/2256; A61B 1/04; A61B 1/00009; A61B 1/00045; A61B 1/06; H04N 5/2257; H04N 5/2258; H04N 5/23219; H04N 5/23293; H04N 5/2624; H04N 2005/2255

(57) ABSTRACT

An endoscope system includes an insertion portion, an observation window provided in the insertion portion and configured to acquire a forward visual field image, an observation window provided in the insertion portion and configured to acquire a lateral visual field image, and an image processing portion. The image processing portion detects a set detection target in the lateral visual field image, generates an image signal of the forward visual field image and an image signal of the lateral visual field image, and in the case that the detection target is detected, outputs the image signal of the forward visual field image and the image signal of the lateral visual field image.

### CROSS REFERENCE TO RELATED APPLICATION

This application is a continuation application of PCT/JP2015/079174 filed on Oct. 15, 2015 and claims benefit of Japanese Application No. 2014-226208 filed in Japan on Nov. 6, 2014, the entire contents of which are incorporated herein by this reference.

### BACKGROUND OF THE INVENTION

1. Field of the Invention

The present invention relates to an endoscope system, and relates in particular to an endoscope system configured to emit illumination light in at least two directions and acquire an object image from the at least two directions.

2. Description of Related Art

Conventionally, an endoscope is widely used in a medical field and an industrial field. The endoscope includes illumination means and observation means on a distal end side of an insertion portion, and is inserted into a subject to observe and inspect an inside of the subject.

In recent years, an endoscope having a wide angle visual field capable of observing two or more directions has been proposed, and as disclosed in Japanese Patent Application Laid-Open Publication No. 2011-152202 and Japanese Patent Application Laid-Open Publication No. 2012-245157 for example, an endoscope apparatus which includes a lateral visual field for which a lateral face side of an insertion portion is an observation visual field in addition to a forward visual field for which a forward side of the insertion portion is an observation visual field, and displays both of a forward visual field image and a lateral visual field image on a monitor has been proposed. Using such an endoscope apparatus, an operator or a tester can simultaneously observe two forward and lateral directions.

### SUMMARY OF THE INVENTION

An endoscope system of one aspect of the present invention includes: an insertion portion configured to be inserted into an inside of a subject; a first image acquisition portion provided in the insertion portion and configured to acquire a main image from a first area; a second image acquisition portion provided in the insertion portion and configured to acquire at least one sub image from a second area including an area different from the first area; an image generation portion configured to generate a first image signal based on the main image and a second image signal based on the sub image; a target detection portion configured to detect a set detection target from the sub image; and an image processing portion configured to output only the first image signal when the detection target is not detected in the target detection portion and output the first image signal and the second image signal when the detection target is detected in the target detection portion.

An endoscope system of one aspect of the present invention includes: an insertion portion configured to be inserted into an inside of a subject; a first image acquisition portion provided in the insertion portion and configured to acquire a main image from a first area; a second image acquisition portion provided in the insertion portion and configured to acquire at least one sub image from a second area including an area different from the first area; an image generation portion configured to generate a first image signal based on the main image and a second image signal based on the sub image; a target detection portion configured to detect a set detection target from the sub image; and an image processing portion configured to output the first image signal and the second image signal when the detection target is detected in the target detection portion and output the first image signal and the second image signal so as to make the main image and the sub image identifiable by lowering luminance of the sub image when the detection target is not detected in the target detection portion.

### BRIEF DESCRIPTION OF THE DRAWINGS

FIG. 1 is a configuration diagram illustrating a configuration of an endoscope system relating to a first embodiment of the present invention;

FIG. 2 is a block diagram illustrating a configuration of an image processing portion 22 relating to the first embodiment of the present invention;

FIG. 3 is a diagram illustrating an example of a detection target setting screen 41 to set a detection target set in a detection target setting portion 32, relating to the first embodiment of the present invention;

FIG. 4 is a diagram illustrating a display state of three display devices 4a, 4b and 4c of a display portion 4 during a predetermined mode, relating to the first embodiment of the present invention;

FIG. 5 is a diagram illustrating the display state of the display portion 4 when a lesioned part PA is detected in a first lateral visual field image, relating to the first embodiment of the present invention;

FIG. 6 is a diagram illustrating another example of the display state of the display portion 4 when the lesioned part PA is detected in the first lateral visual field image, relating to a modification 1 of the first embodiment of the present invention;

FIG. 7 is a diagram illustrating another example of the display state of the display portion 4 when the lesioned part PA is detected in the first lateral visual field image, relating to a modification 2 of the first embodiment of the present invention;

FIG. 8 is a diagram illustrating a display example of three images by a display portion 4A including one display device, relating to a modification 3 of the first embodiment of the present invention;

FIG. 9 is a perspective view of a distal end portion 6a of an insertion portion 6 to which a unit for lateral observation is attached, relating to a modification 4 of the first embodiment of the present invention;

FIG. 10 is a configuration diagram illustrating a configuration of the endoscope system relating to a second embodiment of the present invention;

FIG. 11 is a sectional view of the distal end portion 6a of the insertion portion 6 relating to the second embodiment of the present invention;

FIG. 12 is a block diagram illustrating a configuration of an image processing portion 22A relating the second embodiment of the present invention;

FIG. 13 is a diagram illustrating an example of a display screen of an endoscope image displayed at the display portion 4B, relating the second embodiment of the present invention;

FIG. 14 is a diagram illustrating the display state of the display portion 4B during the predetermined mode, relating the second embodiment of the present invention;

FIG. 15 is a diagram illustrating the display state of the display portion 4B when the lesioned part PA is detected in a lateral visual field image, relating to the second embodiment of the present invention; and

FIG. 16 is a diagram illustrating an example of the display state of the display portion 4B when the lesioned part PA is detected in the lateral visual field image, relating to a modification 2 of the second embodiment of the present invention.

### DETAILED DESCRIPTION OF PREFERRED EMBODIMENTS

Hereinafter, embodiments of the present invention will be described with reference to the drawings.

### First Embodiment

### Configuration

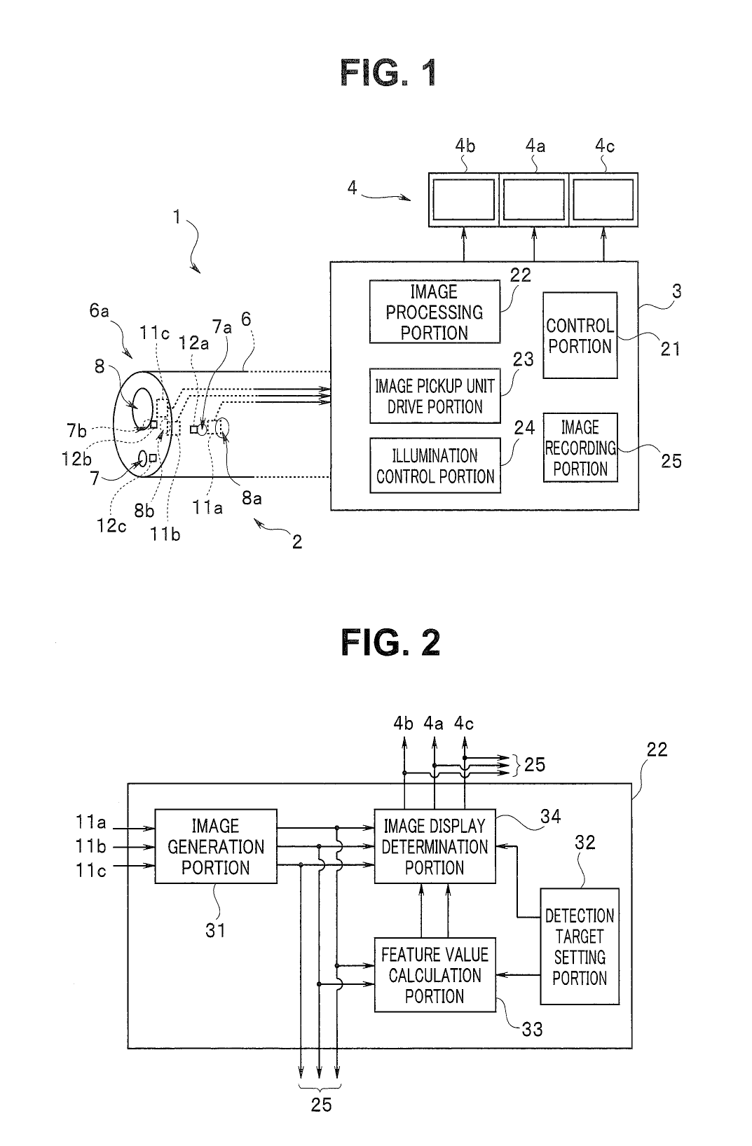

FIG. 1 is a configuration diagram illustrating a configuration of an endoscope system relating to the present embodiment. An endoscope system 1 is configured including an endoscope 2, a processor 3, and a display portion 4.

The endoscope 2 includes an insertion portion 6 configured to be inserted into the inside of a subject and an operation portion not shown in the figure, and is connected to the processor 3 by a cable not shown in the figure. A distal end portion 6a of the insertion portion 6 of the endoscope 2 is provided with an illumination window 7 and an observation window 8 for a forward visual field, and two illumination windows 7a and 7b and two observation windows 8a and 8b for a lateral visual field.

That is, the endoscope 2 includes the two illumination windows 7a and 7b in addition to the illumination window 7, and includes the two observation windows 8a and 8b in addition to the observation window 8. The illumination window 7a and the observation window 8a are for a first lateral visual field, and the illumination window 7b and the observation window 8b are for a second lateral visual field. Then, the plurality of, two in this case, observation windows 8a and 8b are arranged at roughly equal angles in a circumferential direction of the insertion portion 6.

The distal end portion 6a of the insertion portion 6 includes a distal end rigid member not shown in the figure, the illumination window 7 is provided on a distal end face of the distal end rigid member, and the illumination windows 7a and 7b are provided on a lateral face of the distal end rigid member.

On a rear side of the observation window 8a, an image pickup unit 11a for the first lateral visual field is disposed inside the distal end portion 6a, and on a rear side of the observation window 8b, an image pickup unit 11b for the second lateral visual field is disposed inside the distal end portion 6a. On a rear side of the observation window 8 for the forward visual field, an image pickup unit 11c for the forward visual field is disposed.

Each of the three image pickup units 11a, 11b and 11c which are image pickup portions includes an image pickup device, is electrically connected with the processor 3, is controlled by the processor 3, and outputs image pickup signals to the processor 3. The respective image pickup units 11a, 11b and 11c are the image pickup portions that photoelectrically convert an image (object image).

Therefore, the observation window 8 is arranged towards a direction of inserting the insertion portion 6 at the distal end portion 6a of the insertion portion 6, and the observation windows 8a and 8b are arranged towards an outer diameter direction of the insertion portion 6 at a lateral face portion of the insertion portion 6.

That is, the observation window 8 configures a first image acquisition portion provided in the insertion portion 6 and configured to acquire an image of a first object from a forward direction which is a first direction, and each of the observation windows 8a and 8b configures a second image acquisition portion provided in the insertion portion 6 and configured to acquire an image of a second object from a lateral direction which is a second direction different from the forward direction. In other words, the image of the first object is an object image of a first area including an insertion portion forward direction roughly parallel to a longitudinal direction of the insertion portion 6, and the image of the second object is an object image of a second area including an insertion portion lateral direction roughly orthogonal to the longitudinal direction of the insertion portion 6.

The image pickup unit 11c is the image pickup portion that photoelectrically converts the image from the observation window 8, and the image pickup units 11a and 11b are respectively different, that is, separate image pickup portions that photoelectrically convert the two images from the observation windows 8a and 8b.

On a rear side of the illumination window 7a, a light emitting element 12a for illumination for the first lateral visual field is disposed inside the distal end portion 6a, and on a rear side of the illumination window 7b, a light emitting element 12b for the illumination for the second lateral visual field is disposed inside the distal end portion 6a. On a rear side of the illumination window 7 for the forward visual field, a light emitting element 12c for the illumination for the forward visual field is disposed. The light emitting elements 12a, 12b and 12c for the illumination (referred to as the light emitting elements, hereinafter) are light emitting diodes (LEDs) for example.

Therefore, the illumination window 7 corresponding to the light emitting element 12c is an illumination portion that emits illumination light to the forward direction, and the illumination windows 7a and 7b corresponding to each of the light emitting elements 12a and 12b are illumination portions that emit the illumination light to the lateral direction.

The processor 3 includes a control portion 21, an image processing portion 22, an image pickup unit drive portion 23, an illumination control portion 24, and an image recording portion 25.

The control portion 21 includes a central processing unit (CPU), a ROM, a RAM and the like and controls the entire endoscope apparatus.

The image processing portion 22 generates image signals of three endoscope images from the three images obtained based on the three image pickup signals from the three image pickup units 11a, 11b and 11c under control of the control portion 21, converts the image signals to display signals and outputs the display signals to the display portion 4.

Further, the image processing portion 22 performs image processing and setting processing or the like under the control of the control portion 21.

The image pickup unit drive portion 23 is connected with the image pickup units 11a, 11b and 11c by signal lines not shown in the figure. The image pickup unit drive portion 23 drives the image pickup units 11a, 11b and 11c under the control of the control portion 21. The driven image pickup units 11a, 11b and 11c respectively generate the image pickup signals and supply the signals to the image processing portion 22.

The illumination control portion 24 is connected with the light emitting elements 12a, 12b and 12c by signal lines not shown in the figure. The illumination control portion 24 is a circuit that controls the light emitting elements 12a, 12b and 12c under the control of the control portion 21, and controls ON/OFF for each light emitting element. Further, the illumination control portion 24 controls a light quantity of each light emitting element, based on light adjustment signals from the control portion 21.

The image recording portion 25 is a recording portion that records the three endoscope images generated in the image processing portion 22 under the control of the control portion 21, and includes a nonvolatile memory such as a hard disk device.

The display portion 4 includes three display devices 4a, 4b and 4c. To the respective display devices 4a, 4b and 4c, the image signals of the images to be displayed are supplied from the processor 3. A forward visual field image is displayed on a screen of the display device 4a, a first lateral visual field image is displayed on a screen of the display device 4b, and a second lateral visual field image is displayed on a screen of the display device 4c.

The processor 3 is provided with various kinds of operation buttons and a mouse or the like not shown in the figure, and a user operator or the like (referred to as a user, hereinafter) can give to the processor 3 instructions for executing various kinds of functions, that is, instructions for setting an observation mode, recording the endoscope image, and displaying a detection target setting screen to be described later for example.

FIG. 2 is a block diagram illustrating a configuration of the image processing portion 22. The image processing portion 22 includes an image generation portion 31, a detection target setting portion 32, a feature value calculation portion 33, and an image display determination portion 34. To the image processing portion 22, the three image pickup signals from the three image pickup units 11a, 11b and 11c are inputted.

The image generation portion 31 generates the image signals based on the image pickup signals from the respective image pickup units 11a, 11b and 11c, and outputs the respective image signals that are generated to the feature value calculation portion 33 and the image display determination portion 34.

The detection target setting portion 32 is a processing portion that sets a detection target to be detected by image processing in the first lateral visual field image and the second lateral visual field image obtained by picking up the images by the image pickup units 11a and 11b. For example, the detection target is a lesion, a treatment instrument, a lumen, bleeding or the like.

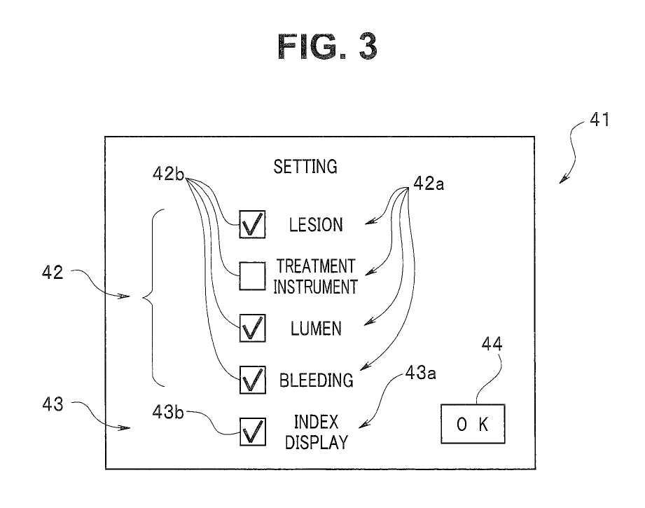

FIG. 3 is a diagram illustrating an example of a detection target setting screen 41 to set the detection target set in the detection target setting portion 32.

The detection target setting screen 41 illustrated in FIG. 3 is displayed on the screen of one of the display devices of the display portion 4 for example by the user operating a predetermined operation button of the processor 3. The user can set the detection target by utilizing the displayed detection target setting screen 41.

The detection target setting screen 41 which is a graphical user interface (GUI) includes a detection target specifying portion 42 which specifies the detection target, an index display setting portion 43 which specifies index display, and an OK button 44 which is a button to instruct completion of setting.

The detection target specifying portion 42 includes a detection target name display portion 42a which indicates the detection target, and a group of a plurality of checkboxes 42b. The user can specify a desired detection target by inputting a checkmark to the checkbox 42b corresponding to a target desired to be detected utilizing the mouse or the like of the processor 3.

For example, FIG. 3 illustrates that “lesion”, “lumen” and “bleeding” are specified as the detection targets since the checkmark is inputted to the checkboxes 42b corresponding to “lesion”, “lumen” and “bleeding”. In a state of FIG. 3, when the user depresses, that is, clicks or the like, the OK button 44, “lesion”, “lumen” and “bleeding” are set to the image processing portion 22 as the detection targets.

When the detection target is set, the detection target setting portion 32 outputs information of the set detection target to the image display determination portion 34, and outputs and instructs information of a feature value to be detected, which is set beforehand for one, two or more detection targets that are set, to the feature value calculation portion 33.

In addition, the index display setting portion 43 includes an index character display portion 43a which displays characters of the index display, and a checkbox 43b for instructing the index display. As described later, the checkbox 43b is for specifying whether or not to display an index indicating a position of the detection target, and by inputting a checkmark in the checkbox 43b, when the set detection target is detected, the index indicating the position of the detected detection target is displayed. That is, the index display setting portion 43 is a setting portion which sets whether or not to display the index at the display portion 4.

Returning to FIG. 2, the feature value calculation portion 33 calculates the feature value to be detected, which is instructed from the detection target setting portion 32, for the respective lateral visual field image signals, and outputs the information of the calculated feature value to the image display determination portion 34.

The feature value calculation portion 33 is capable of calculating the plurality of feature values, calculates the specified feature value, and outputs the value to the image display determination portion 34.

The feature value calculation portion 33 is capable of detecting predetermined color tone, luminance and spatial frequency, presence/absence of an edge, and the like, calculates the feature value specified from the detection target setting portion 32, and outputs the information of the calculated feature value to the image display determination portion 34.

Detection of the predetermined color tone here is color tone detection for detecting whether or not a strongly reddish pixel is present.

Detection of the predetermined luminance here is luminance detection for detecting whether or not a luminal area is present, that is, luminance detection for detecting presence/absence of a dark pixel.

Detection of the predetermined spatial frequency here is spatial frequency detection for detecting presence/absence of a pixel area of the predetermined spatial frequency in order to detect whether or not a lesioned part is present.

Detection of presence/absence of the edge here is edge detection for detecting presence/absence of the pixel area of the edge in order to detect presence/absence of an image of the treatment instrument.

The feature value calculation portion 33 outputs information of a detection result of the pixel or the pixel area having the specified feature value to the image display determination portion 34.

The image display determination portion 34 receives the three image signals from the image generation portion 31, and outputs the forward visual field image to the display device 4a of the display portion 4. For the two lateral visual field images, the image display determination portion 34 judges whether or not to display one or both of the two lateral visual field images at the display portion 4 based on feature value information for the respective images from the feature value calculation portion 33, and outputs one or both of the two lateral visual field images to the display portion 4 based on the judgement result.

Specifically, for the detection target specified by the detection target setting portion 32, the image display determination portion 34 judges whether or not the feature value calculated in the feature value calculation portion 33 satisfies a predetermined condition, and based on the judgement result, judges whether or not to output the display signal for displaying both or one of the two lateral visual field images generated in the image generation portion 31 at the display portion 4.

For example, when the lesion is specified as the detection target, the detection target setting portion 32 outputs information indicating that the detection target is the lesion to the image display determination portion 34, and also outputs information indicating that the feature value to be detected is the predetermined spatial frequency to the feature value calculation portion 33.

The image display determination portion 34 stores judgement reference information such as threshold information for the respective detection targets beforehand Therefore, in the case that the detection target is the lesion, the image display determination portion 34 judges the presence/absence of the lesion based on whether or not a size of the pixel area having the predetermined spatial frequency is equal to or larger than a predetermined threshold TH1.

In addition, when the treatment instrument is specified as the detection target, the detection target setting portion 32 outputs information indicating that the detection target is the treatment instrument to the image display determination portion 34, and also outputs information indicating that the feature value to be detected is the predetermined edge to the feature value calculation portion 33.

Since the treatment instrument is a metal and a surface is glossy and has a color and the luminance completely different from bio-tissue, when the image of the treatment instrument is present in the image, the edge is detected in the image. Therefore, in the case that the detection target is the treatment instrument, the image display determination portion 34 judges the presence/absence of the treatment instrument based on whether or not the pixel area of the predetermined edge is equal to or larger than a predetermined threshold TH2. As a result, for example, when the treatment instrument comes out from a treatment instrument channel, the image of the treatment instrument is displayed at the display portion 4.

Similarly, when the lumen is specified as the detection target, since a luminal part becomes a dark area in the image, the lumen is detected depending on whether or not the pixel area in which the luminance is equal to or lower than a threshold TH3 is equal to or larger than a predetermined threshold TH4.

In addition, when the bleeding is specified as the detection target, the bleeding is detected depending on whether or not a red pixel area is equal to or larger than a predetermined threshold TH5.

Note that, here, the feature values of the luminance, the spatial frequency, the color and the edge of the pixel or the pixel area are used for the detection of the detection target; however, the other features values may be used.

Therefore, the feature value calculation portion 33 and the image display determination portion 34 configure a target detection portion configured to detect the set detection target by image processing in the respective lateral visual field images.

When the set detection target is detected, the image display determination portion 34 outputs the image signal of the lateral visual field image including the detection target to the display portion 4.

That is, the image generation portion 31 and the image display determination portion 34 generate the image signal of the forward visual field image and the image signals of the two lateral visual field images, and in the case that the detection target is detected in the feature value calculation portion 33 and the image display determination portion 34, convert the image signal of the forward visual field image and the image signal of the lateral visual field image in which the detection target is detected to the display signals and output the display signals to the display portion 4. As a result, the forward visual field image is displayed at the display device 4a of the display portion 4, and the lateral visual field image in which the detection target is detected is displayed at the display device 4b or the display device 4c.

In addition, the image recording portion 25 is a processing portion which records the endoscope image during an inspection, and when the inspection is started, records one, two or more images judged in the image display determination portion 34 and displayed in the display portion 4, and also records the three images generated in the image generation portion 31, that is, the forward visual field image and the first and second lateral visual field images.

Here, since the three images generated in the image generation portion 31 are also recorded in the image recording portion 25 in addition to the one or more images displayed at the display portion 4, that is, the forward visual field image and the one or two lateral visual field images in which the detection target is detected, all the images during the inspection can be played back and viewed again after the inspection so that occurrence of an oversight of the lesion or the like is prevented.

Note that the image recording portion 25 may record either one, two or more images displayed at the display portion 4 or all the images generated in the image generation portion 31.

### (Action)

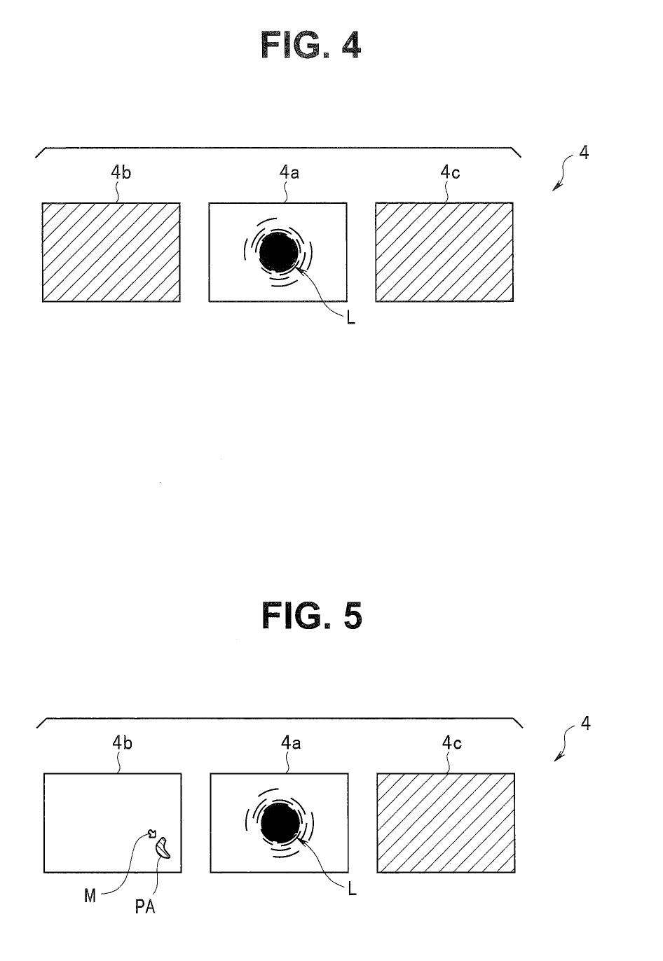

FIG. 4 is a diagram illustrating a display state of the three display devices 4a, 4b and 4c of the display portion 4 during a predetermined mode.

When the user sets the endoscope system 1 to the predetermined mode, first, only the forward visual field image is displayed at the display device 4a, and the first lateral visual field image and the second lateral visual field image are not displayed at the display devices 4b and 4c as indicated by oblique lines in FIG. 4. In FIG. 4, the user inserts the insertion portion into a large intestine and performs the inspection, and a lumen L is displayed in the forward visual field image.

When outputting only the image signal of the forward visual field image, the image processing portion 22 detects the presence/absence of the detection target in the first lateral visual field image and the second lateral visual field image. When the detection target set in the detection target setting portion 32 is not detected in the first lateral visual field image and the second lateral visual field image, the image processing portion 22 outputs only the image signal of the forward visual field image.

That is, when the image processing portion 22 outputs only the image signal of the forward visual field image in the case that the detection target is not detected, the image processing portion 22 detects the presence/absence of the detection target in the first lateral visual field image and the second lateral visual field image.

When the detection target set in the detection target setting portion 32 described above is detected in the first or second lateral visual field image other than the forward visual field image, the lateral visual field image including the detected detection target is displayed at the corresponding display device.

FIG. 5 is a diagram illustrating the display state of the display portion 4 when a lesioned part PA is detected in the first lateral visual field image.

When the lesion is detected when “lesion”, “lumen” and “bleeding” are set as the detection targets as illustrated in FIG. 3 for example in the detection target setting portion 32, the lateral visual field image including the lesioned part PA is displayed at the display portion 4.

FIG. 5 illustrates that the first lateral visual field image is displayed at the display device 4b without any display until then. Further, since the index display is also set as illustrated in FIG. 3, an index M which is an arrow mark is displayed near the detected lesioned part PA.

That is, when outputting the image signal of the lateral visual field image, the image processing portion 22 outputs index information for displaying the index M indicating the position of the detection target in the lateral visual field image at the corresponding display device 4b or 4c of the display portion 4.

While the user executes an intraluminal inspection while advancing the distal end portion 6a of the insertion portion 6 in an inserting direction or a removing direction, normally, the forward visual field image is displayed at the display device 4a of the display portion 4, and only the forward visual field image is looked carefully and observed. When the set detection target such as the lesion is detected by the image processing, the lateral visual field image including the detection target is displayed at the corresponding display device 4b or 4c of the display portion 4. When the set detection target is not detected, the inspection can be performed looking at only the forward visual field image, that is, paying attention to the forward visual field image only, so that the user is not required to look at all the three images and can quickly advance the inspection with less burden.

However, when the set detection target is detected in at least one of the two lateral visual field images, the lateral visual field image including the detected detection target is displayed at the display portion 4.

As described above, the two lateral visual field images are present, and the image processing portion 22 outputs the image signal of the forward visual field image and the image signals of the two lateral visual field images so as to arrange the forward visual field image at a center and display the two lateral visual field images to sandwich the forward visual field image at the display portion 4, and when the detection target is detected in one of the two lateral visual field images, outputs the image signal of the lateral visual field image so as to display only the lateral visual field image in which the detection target is detected.

Therefore, in the case that the set detection target is detected, since the user can also look at the one or two lateral visual field images, the lesion can be confirmed by looking at the newly displayed lateral visual field image. Since the user needs to carefully look at the two or three images only in the case that the set detection target is detected, the inspection can be quickly performed with less burden in the entire inspection.

As described above, according to the above-described embodiment, the endoscope system capable of reducing the burden on an operator at a time when the operator observes the endoscope image of a wide angle visual field can be provided.

As a result, an oversight of a part to be observed such as the lesion can be prevented.

In the present embodiment and the other embodiment described later, an image of the first object (a first object image, the forward visual field image) from the forward direction which is the first direction is defined as a main image which is an image to be mainly displayed since it is demanded to be observed almost all the time during an operation of the endoscope system 1.

In addition, an image of the second object (a second object image, the lateral visual field image) from the lateral direction which is the second direction is defined as a sub image since it is not always needed to be displayed mainly in contrast with the above-described main image.

Note that, based on the above-described definitions of the main image and the sub image, in a lateral view type endoscope, a main observation window of which is turned to the lateral direction of the insertion portion 6 all the time for example, in the case of arranging a simple observation window turned to the forward direction in order to improve insertion to the forward direction which is an insertion axis direction, the lateral visual field image may be defined as the main image, the lateral visual field image may be defined as the sub image, and the processing according to the above-described first embodiment may be performed.

That is, an area (first direction) to acquire the main image may be one of an area including the insertion portion forward direction roughly parallel to the longitudinal direction of the insertion portion and an area including the insertion portion lateral direction roughly orthogonal to the longitudinal direction of the insertion portion, and an area (second direction) to acquire the sub image may be the other of the insertion portion forward direction and the insertion portion lateral direction.

### (Modification 1)

In the above-described embodiment, when the detection target is detected, the lateral visual field image including the detection target is displayed at the display portion 4; however, the lateral visual field image not including the detection target may be also displayed.

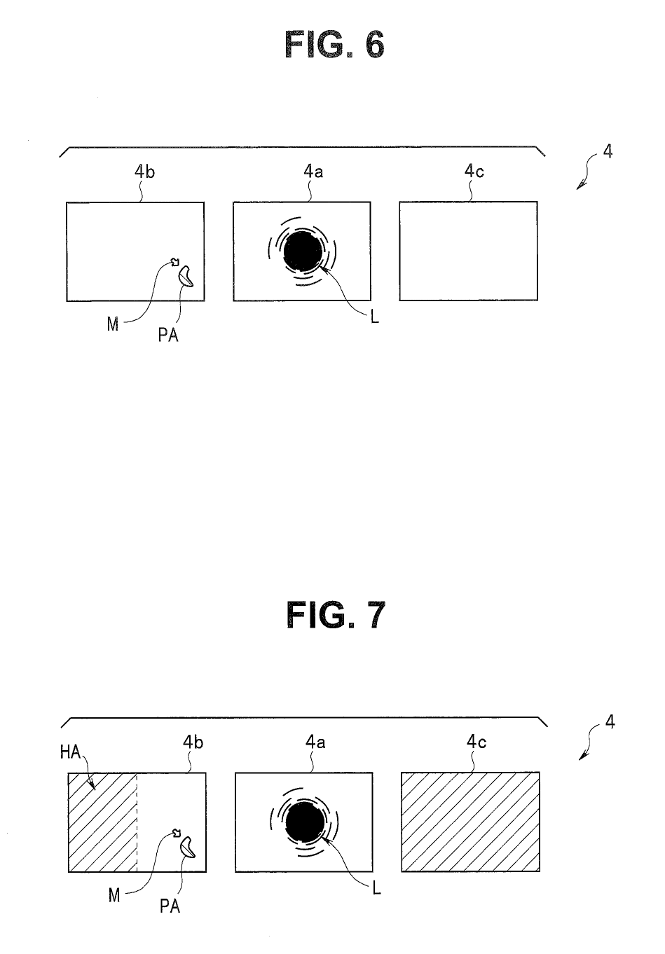

FIG. 6 is a diagram illustrating another example of the display state of the display portion 4 when the lesioned part PA is detected in the first lateral visual field image, relating to the modification 1.

At the display portion 4 in FIG. 6, when the lesioned part PA is detected in the first lateral visual field image, not only the first lateral visual field image in which the lesioned part PA is detected but also the second lateral visual field image in which the lesioned part PA is not detected is displayed.

That is, when some detection target is displayed, since it is sometimes desired to confirm the image of a peripheral area as well, the two lateral visual field images may be displayed as in FIG. 6.

Note that, in this case, in order to easily identify the lateral visual field image including the detection target and the lateral visual field image not including the detection target, display may be performed while making the luminance of the lateral visual field image not including the detection target lower than the luminance of the lateral visual field image including the detection target.

### (Modification 2)

In the above-described embodiment, when the detection target is detected, the entire lateral visual field image including the detection target is displayed at the display portion 4; however, only an image area near the detection target in the lateral visual field image may be displayed.

FIG. 7 is a diagram illustrating another example of the display state of the display portion 4 when the lesioned part PA is detected in the first lateral visual field image, relating to the modification 2.

When the lesioned part PA is detected in the first lateral visual field image, at the display portion 4 in FIG. 7, only a half area including the area in which the lesioned part PA is detected in the first lateral visual field image in which the lesioned part PA is detected is displayed.

That is, the image display determination portion 34 of the image processing portion 22 converts the image signal for displaying a part of the first lateral visual field image in which the lesioned part PA is detected into the display signal and outputs the signal to the display device 4b. As a result, when the set detection target is displayed, in order to allow the user to visually recognize the detection target quickly in the lateral visual field image including the detection target, an area HA other than the image area including the detection target is not displayed.

Note that, in this case, display may be performed while making the luminance of the area HA other than the image area including the detection target lower than the luminance of the image area including the detection target.

### (Modification 3)

In the embodiment and the modifications 1 and 2 described above, the display portion 4 is configured from the three display devices; however, the three images may be displayed at one display device.

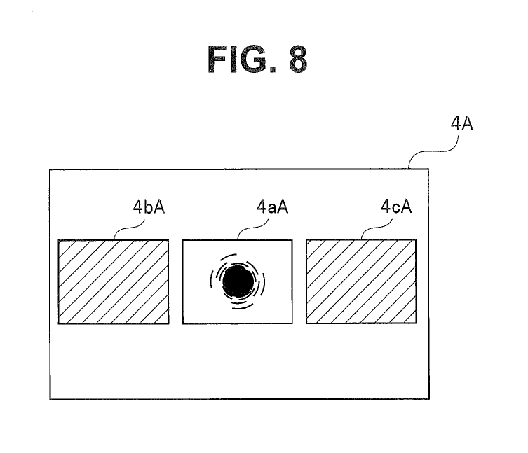

FIG. 8 is a diagram illustrating a display example of the three images by a display portion 4A including one display device, relating to the modification 3. The display portion 4A is formed of one display device, and the three images, that is, a forward visual field image 4aA and two lateral visual field images 4bA and 4cA respectively corresponding to the forward visual field image 4a and the two lateral visual field images 4b and 4c in FIG. 4 described above, are displayed on one screen of the display device.

The three endoscope images can be displayed in a display form as described above also in FIG. 8.

### (Modification 4)

In the embodiment and the respective modifications described above, a mechanism that realizes a function of illuminating and observing the lateral direction is built in the insertion portion 6 together with a mechanism that realizes a function of illuminating and observing the forward direction; however, the mechanism that realizes the function of illuminating and observing the lateral direction may be a separate body attachable and detachable to/from the insertion portion 6.

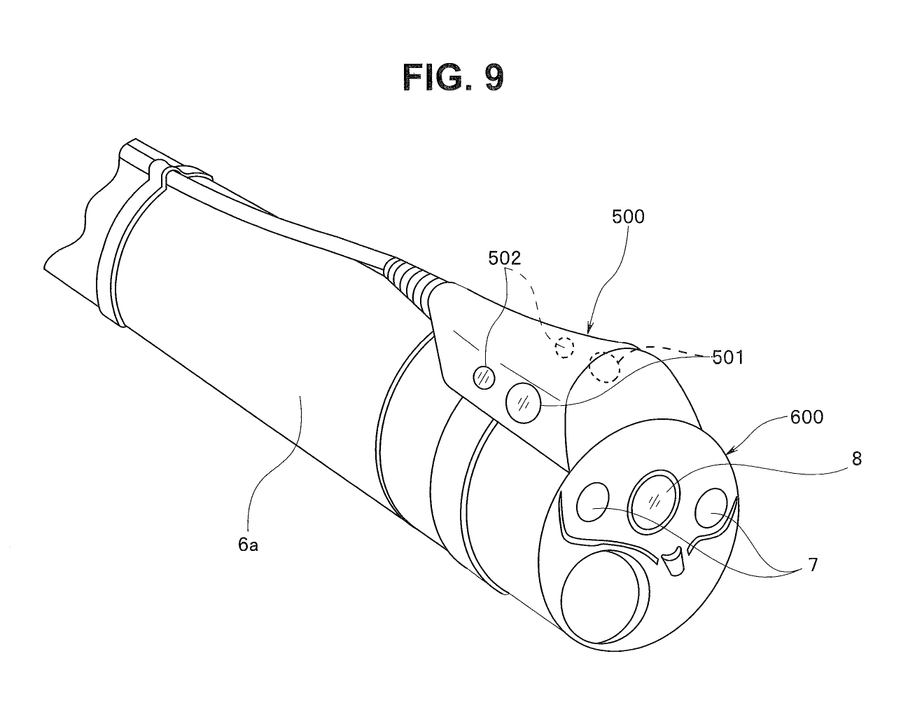

FIG. 9 is a perspective view of the distal end portion 6a of the insertion portion 6 to which a unit for lateral observation is attached. The distal end portion 6a of the insertion portion 6 includes a unit 600 for the forward visual field. A unit 500 for the lateral visual field has a configuration freely attachable and detachable to/from the unit 600 for the forward visual field.

The unit 500 for the lateral visual field includes two observation windows 501 for acquiring images in left and right directions, and two illumination windows 502 for illuminating the left and right directions.

The processor 3 or the like can acquire and display observation images as indicated in the above-described embodiment by lighting and putting out the respective illumination windows 502 of the unit 500 for the lateral visual field in accordance with a frame rate of the forward visual field.

As described above, according to the embodiment and the respective modifications described above, the endoscope system capable of reducing the burden on an operator at a time when the operator observes the endoscope image of a wide angle visual field can be provided.

As a result, an oversight of a part to be observed such as the lesion can be prevented.

Further, for preservation of the endoscope images, since both of the displayed image and all the images are preserved, an oversight can be prevented even in the case of reviewing the images later.

### Second Embodiment

Two or more image pickup devices are built in the distal end portion 6a of the insertion portion 6 of the endoscope in the first embodiment in order to acquire the object images from at least two directions; however, one image pickup device is built in the distal end portion 6a of the insertion portion 6 of the endoscope in the present embodiment in order to acquire the object images from at least two directions.

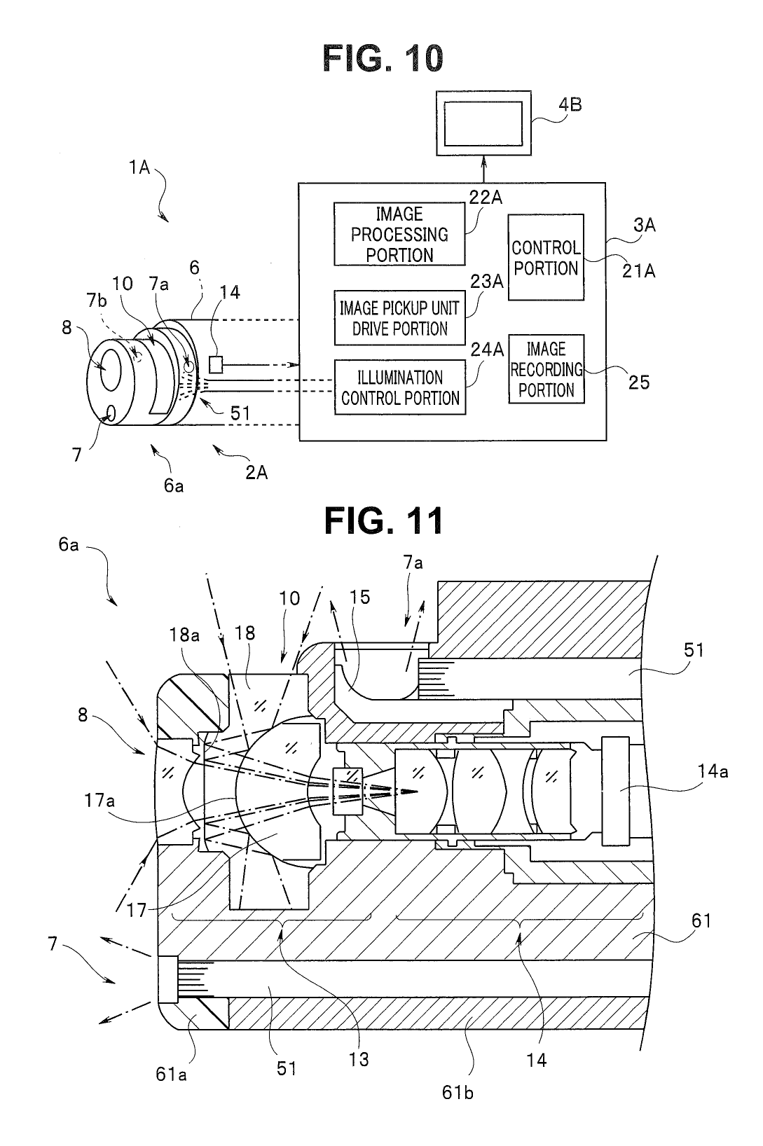

FIG. 10 is a configuration diagram illustrating a configuration of the endoscope system relating to the present embodiment. Since the endoscope system 1A in the present embodiment includes the configuration almost similar to that of endoscope system 1 in the first embodiment, same signs are attached and descriptions are omitted for components same as those of the endoscope system 1, and different configurations will be described.

The distal end portion 6a of the insertion portion 6 of an endoscope 2A is provided with the illumination window 7 and the observation window 8 for the forward visual field, and two illumination windows 7a and 7b and an observation window 10 for the lateral visual field. The observation window 10 which is an image acquisition portion is arranged closer to a proximal end side of the insertion portion 6 than the observation window 8 which is the image acquisition portion.

In addition, for illumination, a light guide 51 formed of an optical fiber bundle is used instead of the light emitting element. On a proximal end portion of the light guide 51, illumination light for the three illumination windows 7, 7a and 7b is incident. A distal end portion of the light guide 51 is equally divided into three and arranged on the rear side of the three illumination windows 7, 7a and 7b.

FIG. 11 is a sectional view of the distal end portion 6a of the insertion portion 6. Note that FIG. 11 illustrates a cross section for which the distal end portion 6a is cut so as to recognize cross sections of the illumination window 7a for the lateral visual field, the illumination window 7 for the forward illumination and the observation window 8 for the forward visual field.

On the rear side of the illumination window 7, a distal end face of a part of the light guide 51 is disposed. The observation window 8 is provided on a distal end face of a distal end rigid member 61. On the rear side of the observation window 8, an objective optical system 13 is disposed.

On the rear side of the objective optical system 13, an image pickup unit 14 is disposed. Note that, to the distal end portion of the distal end rigid member 61, a cover 61a is attached. In addition, a jacket 61b is put on the insertion portion 6.

Therefore, the illumination light for the forward direction is emitted from the illumination window 7, and reflected light from an object which is an observation part inside a subject is incident on the observation window 8.

The two illumination windows 7a and 7b are disposed on a lateral face of the distal end rigid member 61, and behind the respective illumination windows 7a and 7b, the distal end face of a part of the light guide 51 is disposed through a mirror 15, a reflection surface of which is a curved surface.

Therefore, the illumination window 7 and the plurality of illumination windows 7a and 7b configure an illumination light emission portion which emits first illumination light to a forward area as the first area and emits second illumination light to a lateral area as the second area different from the first area inside the subject.

The second area different from the first area indicates an area of a visual field in a direction in which an optical axis is turned to a different direction, and the first area (first object image) and the second area (second object image) may or may not partially overlap, and further, an irradiation range of the first illumination light and an irradiation range of the second illumination light may or may not partially overlap.

The observation window 10 is disposed on the lateral face of the distal end rigid member 61, and the objective optical system 13 is disposed on the rear side of the observation window 10. The objective optical system 13 is configured to turn the reflected light from the forward direction, which passes through the observation window 8, and the reflected light from the lateral direction, which passes through the observation window 10, to the image pickup unit 14. In FIG. 11, the objective optical system 13 includes two optical members 17 and 18. The optical member 17 is a lens including a convex surface 17a, and the optical member 18 includes a reflection surface 18a which causes light from the convex surface 17a of the optical member 17 to reflect towards the image pickup unit 14 through the optical member 17.

That is, the observation window 8 configures the first image acquisition portion provided in the insertion portion 6 and configured to acquire an image of the first object from the forward direction which is the first area, and the observation window 10 configures the second image acquisition portion provided in the insertion portion 6 and configured to acquire an image of the second object from the lateral direction which is the second area different from the forward direction.

More specifically, the image from the forward area which is the first area is the object image of the first area including the forward direction of the insertion portion 6 roughly parallel to the longitudinal direction of the insertion portion 6, the image from the lateral area which is the second area is the object image of the second area including the lateral direction of the insertion portion 6 roughly orthogonal to the longitudinal direction of the insertion portion 6, the observation window 8 is a forward image acquisition portion which acquires the object image of the first area including the forward direction of the insertion portion 6, and the observation window 10 is a lateral image acquisition portion which acquires the object image of the second area including the lateral direction of the insertion portion 6.

Then, the observation window 8 which is the image acquisition portion is arranged at the distal end portion 6a of the insertion portion 6 towards the direction of inserting the insertion portion 6, and the observation window 10 which is the image acquisition portion is arranged at the lateral face portion of the insertion portion 6 towards the outer diameter direction of the insertion portion 6. The image pickup unit 14 which is the image pickup portion is arranged so as to photoelectrically convert the object image from the observation window 8 and the object image from the observation window 10 on the same image pickup surface, and is electrically connected to the processor 3 including the image processing portion 22.

That is, the observation window 8 is arranged at the distal end portion in the longitudinal direction of the insertion portion 6 so as to acquire the first object image from the direction of inserting the insertion portion 6, and the observation window 10 is arranged along the circumferential direction of the insertion portion 6 so as to acquire the second object image from the second direction. Then, the image pickup unit 14 electrically connected with the processor 3 photoelectrically converts the first object image and the second object image on one image pickup surface, and supplies the image pickup signals to the processor 3.

Therefore, the illumination light for the forward direction is emitted from the illumination window 7, the reflected light from the object passes through the observation window 8 and is incident on the image pickup unit 14, the illumination light for the lateral direction is emitted from the two illumination windows 7a and 7b, and the reflected light from the object passes through the observation window 10 and is incident on the image pickup unit 14. An image pickup device 14a of the image pickup unit 14 photoelectrically converts an optical image of the object, and outputs the image pickup signal to a processor 3A.

Returning to FIG. 10, the image pickup signal from the image pickup unit 14 is supplied to the processor 3A which is the image generation portion, and the endoscope image is generated. The processor 3A converts the signal of the endoscope image which is the observation image to the display signal and outputs the signal to a display portion 4B.

The processor 3A includes a control portion 21A, an image processing portion 22A, an image pickup unit drive portion 23A, an illumination control portion 24A, and the image recording portion 25.

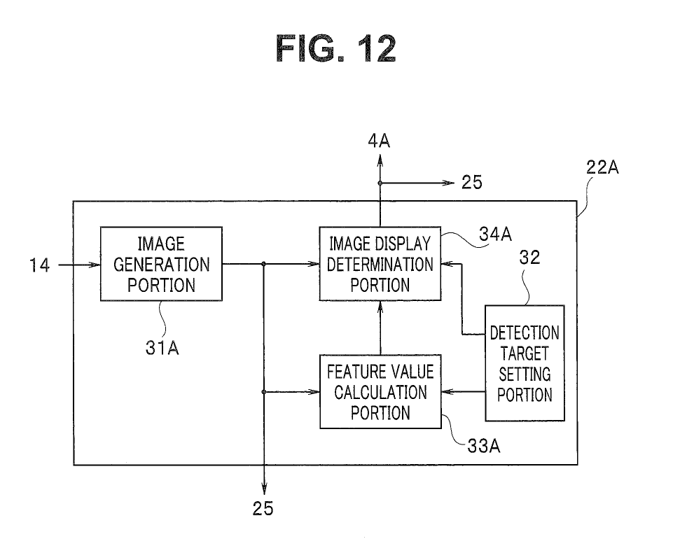

FIG. 12 is a block diagram illustrating a configuration of the image processing portion 22A. The image processing portion 22A includes an image generation portion 31A, the detection target setting portion 32, a feature value calculation portion 33A, and an image display determination portion 34A. To the image processing portion 22A, the image pickup signal from the image pickup unit 14 is inputted.

The image generation portion 31A has the function similar to that of the image generation portion 31 described above, generates the image signal based on the image pickup signal from the image pickup unit 14, and outputs the generated image signal to the feature value calculation portion 33A and the image display determination portion 34A.

The detection target setting portion 32 is in the configuration similar to that of the first embodiment, and is a processing portion which sets the detection target to be detected by the image processing in the lateral visual field image obtained by picking up the image by the image pickup unit 14 by a setting screen as illustrated in FIG. 3.

Returning to FIG. 12, the feature value calculation portion 33A calculates the feature value to be detected, which is instructed from the detection target setting portion 32, for the lateral visual field image signal, and outputs the information of the calculated feature value to the image display determination portion 34A.

The feature value calculation portion 33A has the function similar to that of the feature value calculation portion 33 described above, calculates the feature value specified from the detection target setting portion 32 in the lateral visual field image, and outputs the information of the calculated feature value to the image display determination portion 34A.

The image display determination portion 34A has the function similar to that of the image display determination portion 34 described above, receives the image from the image generation portion 31A, converts the forward visual field image to the display signal and outputs the display signal to the display portion 4B of the display portion 4 at all times. For the lateral visual field image, the image display determination portion 34A judges whether or not to display the lateral visual field image at the display portion 4B based on the feature value information for the image from the feature value calculation portion 33A, and based on the judgement result, converts the lateral visual field image to the display signal and outputs the display signal to the display portion 4B.

When the set detection target is detected, the image display determination portion 34A causes the lateral visual field image to be displayed at the display portion 4B.

That is, when the detection target set in the detection target setting portion 32 is detected in the lateral visual field image, the image display determination portion 34A displays the lateral visual field image at the display portion 4B together with the forward visual field image.

In addition, when the detection target set in the detection target setting portion 32 is not detected in the lateral visual field image, the image display determination portion 34A does not display the lateral visual field image, magnifies the forward visual field image, and displays the image at the display portion 4B.

An operation of the image recording portion 25 is similar to that of the first embodiment.

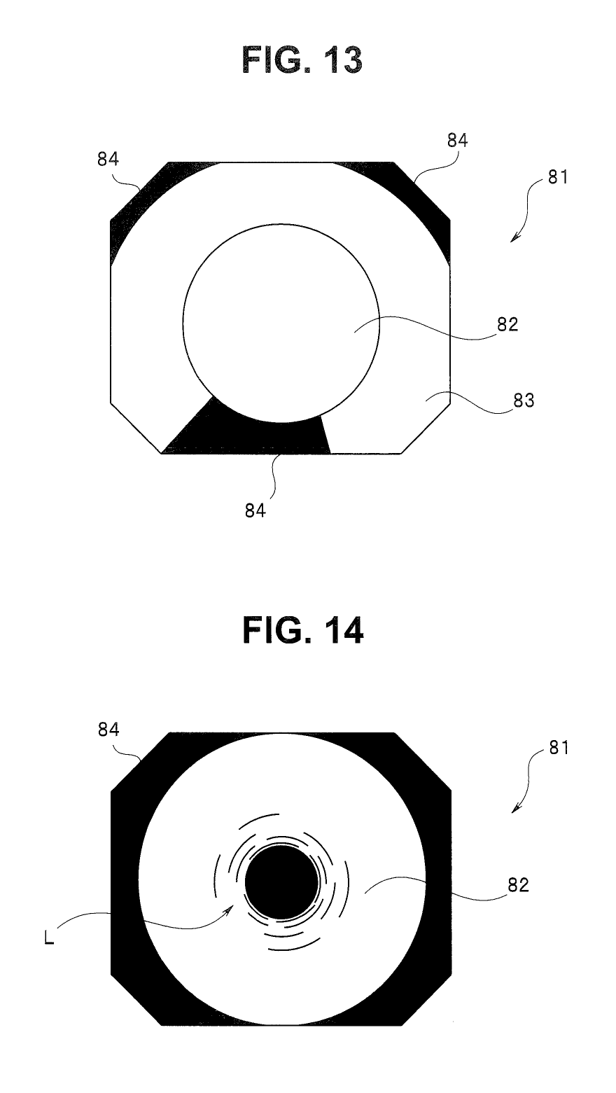

FIG. 13 is a diagram illustrating an example of a display screen of the endoscope image displayed at the display portion 4B, relating the present embodiment.

A display image 81 which is the endoscope image displayed on the screen of the display portion 4 is a roughly rectangular image, and includes two areas 82 and 83. The circular area 82 at a center portion is an area that displays the forward visual field image, and the C-shaped area 83 around the area 82 at the center portion is an area that displays the lateral visual field image. FIG. 13 illustrates the state when both of the forward visual field image and the lateral visual field image are displayed, and the image processing portion 22A outputs the image signal of the forward visual field image and the image signal of the lateral visual field image such that the lateral visual field image is displayed around the forward visual field image at the display portion 4B.

That is, the forward visual field image is displayed on the screen of the display portion 4 so as to be roughly circular, and the lateral visual field image is displayed on the screen so as to be roughly annular surrounding at least a part of a circumference of the forward visual field image. Therefore, at the display portion 4, the wide angle endoscope image is displayed.

The endoscope image illustrated in FIG. 13 is generated from an acquisition image acquired by the image pickup device 14a. The forward visual field image and the lateral visual field image are cut and generated from the image obtained in the image pickup device 14a. The display image 81 is generated by photoelectrically converting the object image projected to the image pickup surface of the image pickup device 14a by the optical system illustrated in FIG. 11, and compositing a forward visual field image region at the center corresponding to the area 82 and the lateral visual field image area corresponding to the area 83 excluding an area 84 painted out black as a mask area.

### (Action)

FIG. 14 is a diagram illustrating the display state of the display portion 4B during a predetermined mode.

When the user sets the endoscope system 1 to the predetermined mode, first, the area 82 is cut from the image obtained by picking up the image in the image pickup device 14a and is magnified and displayed at the display portion 4B, and the lateral visual field image is not displayed. If the user is performing the inspection by inserting the insertion portion into the large intestine for example, the lumen L is displayed in the forward visual field image.

However, when the detection target set in the detection target setting portion 32 described above is detected in the lateral visual field image, the lateral visual field image including the detected detection target is displayed at the display portion 4B.

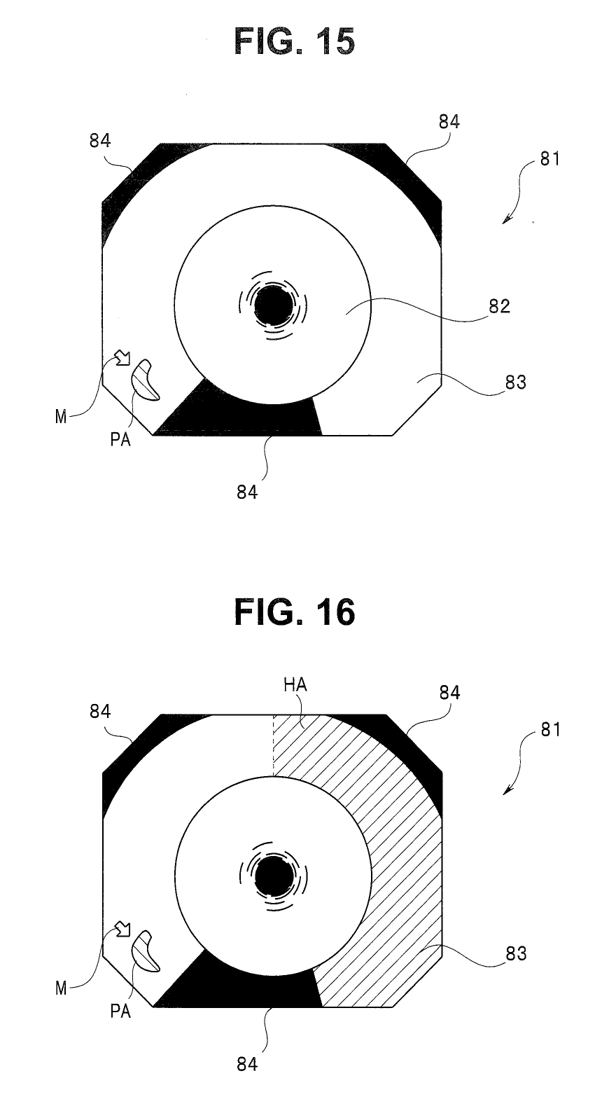

FIG. 15 is a diagram illustrating the display state of the display portion 4B when the lesioned part PA is detected in the lateral visual field image.

Similarly to the first embodiment, in the detection target setting portion 32, as illustrated in FIG. 3, when “lesion”, “lumen” and “bleeding” are set as the detection targets and the index display is also set, when the lesion is detected, the lateral visual field image including the lesioned part PA is displayed at the display portion 4 together with the index M. In FIG. 15, the forward visual field image is not magnified as in FIG. 14.

That is, while the user executes the intraluminal inspection while advancing the distal end portion of the insertion portion in the inserting direction or the removing direction, normally, the forward visual field image is displayed at the display portion 4B, and only the forward visual field image is looked carefully and observed. When the set detection target such as the lesion is detected by the image processing, the lateral visual field image including the detection target is displayed at the display portion 4B.

When the set detection target is not detected, the inspection can be performed looking at only the magnified forward visual field image, that is, paying attention to the forward visual field image only, so that the user is not required to look at both images of the forward visual field image and the lateral visual field image and can quickly advance the inspection with less burden.

However, when the set detection target is detected in the lateral visual field image, the lateral visual field image including the detected detection target is displayed at the display portion 4B. In the case that the set detection target is detected, since the user can look at the lateral visual field image as well, the lesion can be confirmed by looking at the newly displayed lateral visual field image. Since the user needs to carefully look at the lateral visual field image only in the case that the set detection target is detected, the inspection can be quickly performed with less burden in the entire inspection.

As described above, according to the above-described embodiment, the endoscope system capable of reducing the burden on an operator at a time when the operator observes the endoscope image of a wide angle visual field can be provided.

As a result, an oversight of a part to be observed such as the lesion can be prevented.

### (Modification 1)

In the above-described second embodiment, the forward visual field image is magnified and displayed when the lateral visual field image is not displayed; however, the forward visual field image may be displayed without being magnified.

### (Modification 2)

In the above-described second embodiment, when the detection target is detected, the entire lateral visual field image including the detection target is displayed at the display portion 4B; however, only the image area near the detection target in the lateral visual field image may be displayed.

FIG. 16 is a diagram illustrating an example of the display state of the display portion 4B when the lesioned part PA is detected in the lateral visual field image, relating to the modification 2.

When the lesioned part PA is detected in the lateral visual field image, at the display portion 4B in FIG. 16, only the half area including the area in which the lesioned part PA is detected in the lateral visual field image in which the lesioned part PA is detected is displayed.

That is, when some detection target is displayed, in order to allow the user to visually recognize the detection target quickly in the lateral visual field image including the detection target, the area HA other than the image area including the detection target is not displayed.

Note that, in this case, display may be performed while making the luminance of the area HA other than the image area including the detection target lower than the luminance of the image area including the detection target.

As described above, according to the second embodiment and the respective modifications described above, the endoscope system capable of reducing the burden on an operator at a time when the operator observes the endoscope image of a wide angle visual field can be provided.

As a result, an oversight of a part to be observed such as the lesion can be prevented.

Further, for preservation of the endoscope images, since both of the displayed image and all the images are preserved, an oversight can be prevented even in the case of reviewing the images later.

Note that, in the two embodiments described above, when the detection target is detected, the index is displayed near the detection target; however, the display of the index may be set for each detection target. That is, the index may be displayed when the lesion is detected and the index may not be displayed when the treatment instrument is detected.

Further, note that, in the respective embodiments described above, the lateral visual field image is not displayed when the detection target is not detected; however, the lateral visual field image may be displayed darkly by applying a gray mask or the like to the lateral visual field image when the detection target is not detected.

That is, when the detection target is not detected, the image processing portions 22 and 22A may output the image signal of the forward visual field image and the image signal of the lateral visual field image so as to make the forward visual field image and the lateral visual field image identifiable by lowering the luminance of the lateral visual field image.

In addition, in the respective embodiments, the detection target is detected based on the lateral image (sub image, second image) of the image signal generated based on the image pickup signal generated from the image pickup unit; however, the detection target may be detected directly from the image pickup signals relating to the lateral direction (second area) generated from the image pickup unit.

The present invention is not limited to the embodiments described above, and can be variously modified or altered or the like without changing the scope of the present invention.

What is claimed is:

1. An endoscope system comprising:
an insertion portion configured to be inserted into an inside of a subject;
a first image acquisition portion provided in the insertion portion and configured to acquire a main image from a first area;
a second image acquisition portion provided in the insertion portion and configured to acquire at least one sub image from a second area including an area different from the first area;
an image generation portion configured to generate a first image signal based on the main image and a second image signal based on the sub image;
a target detection portion configured to detect a set detection target from the sub image; and
an image processing portion configured to output only the first image signal when the detection target is not detected in the target detection portion and output the first image signal and the second image signal when the detection target is detected in the target detection portion.

2. The endoscope system according to claim 1,
wherein the first area is an area including an insertion portion forward direction roughly parallel to a longitudinal direction of the insertion portion, and
the second area is an area including an insertion portion lateral direction roughly orthogonal to the longitudinal direction of the insertion portion.

3. The endoscope system according to claim 1,
wherein, when the image processing portion outputs only the first image signal based on the main image in a case that the detection target is not detected in the target detection portion, the target detection portion detects presence/absence of the detection target in the sub image.

4. The endoscope system according to claim 1, comprising a detection target setting portion configured to set the detection target to be detected in the target detection portion.

5. The endoscope system according to claim 4,
wherein the detection target is at least one of a lesion, a treatment instrument, a lumen and bleeding.

6. The endoscope system according to claim 1,
wherein the image processing portion outputs only the first image signal when the detection target is not detected in the target detection portion and outputs a part of the second image signal and the first image signal when the detection target is detected in the target detection portion.

7. The endoscope system according to claim 1,
wherein the image processing portion converts the first image signal or both of the first image signal and the second image signal into a display signal, and outputs the display signal to a display portion configured to display images.

8. The endoscope system according to claim 7,
wherein the image processing portion outputs the first image signal and the second image signal so as to arrange the main image at a center and display two of the sub images to sandwich the main image at the display portion, and when the detection target is detected in one of the two sub images in the target detection portion, outputs the second image signal so as to display only the sub image in which the detection target is detected.

9. The endoscope system according to claim 7,
wherein the second image acquisition portion for acquiring the sub image is arranged in plurality at roughly equal angles in a circumferential direction of the insertion portion, and
the image processing portion outputs the first image signal and the second image signal so as to arrange the main image at a center and display two of the sub images to sandwich the main image at the display portion.

10. The endoscope system according to claim 9,
wherein the first image acquisition portion includes a first image pickup portion configured to photoelectrically convert the main image, and
the second image acquisition portion includes a second image pickup portion different from the first image pickup portion configured to photoelectrically convert the sub images.

11. The endoscope system according to claim 7,
wherein the image processing portion outputs the first image signal and the second image signal so as to display the sub image around the main image at the display portion.

12. The endoscope system according to claim 11,
wherein the first image acquisition portion is arranged at a distal end portion in a longitudinal direction of the insertion portion so as to acquire the main image from a first direction which is a direction of inserting the insertion portion, and
the second image acquisition portion is arranged along a circumferential direction of the insertion portion so as to acquire the sub image from a second direction.

13. The endoscope system according to claim 7, comprising
an image pickup portion configured to photoelectrically convert the main image from the first image acquisition portion and the sub image from the second image acquisition portion on one image pickup surface,
wherein the image generation portion generates image signals including the first image signal based on the main image and the second image signal based on the sub image.

14. The endoscope system according to claim 13,
wherein the image processing portion outputs the first and second image signals so as to display the sub image at at least a part of a circumference of the main image at the display portion.

15. An endoscope system comprising:
an insertion portion configured to be inserted into an inside of a subject;
a first image acquisition portion provided in the insertion portion and configured to acquire a main image from a first area;
a second image acquisition portion provided in the insertion portion and configured to acquire at least one sub image from a second area including an area different from the first area;
an image generation portion configured to generate a first image signal based on the main image and a second image signal based on the sub image;
a target detection portion configured to detect a set detection target from the sub image; and
an image processing portion configured to output the first image signal and the second image signal when the detection target is detected in the target detection portion and output the first image signal and the second image signal so as to make the main image and the sub image identifiable by lowering luminance of the sub image when the detection target is not detected in the target detection portion.
